(19) United States
(12) Patent Application Publication
(10) Pub. No.: US 2026/0258327 A1
(43) Pub. Date: Sep. 3, 2026

(54) AN ACOUSTIC TREATMENT METHOD AND DEVICE

(71) Applicant: CRAFT BREWING SOLUTIONS PTY LTD, Mentone, Victoria (AU)

(72) Inventors: Boon Mian TEO, Mentone,Victoria (AU); David Jonathan BROUGH, Mentone, Victoria (AU)

(21) Appl. No.: 19/491,231

(22) PCT Filed: Jun. 28, 2024
(86) PCT No.: PCT/AU2024/050701

§ 371 Date: Dec. 8, 2025

(30) Foreign Application Priority Data
Jun. 28, 2023 (AU) 2023902036

(51) Int. Cl. C12C 11/00; C12M 1/42; C12N 13/00

(52) U.S. Cl. CPC C12C 11/003; C12M 35/04; C12N 13/00

(57) ABSTRACT

A method of culturing a microorganism, the method including: (i) providing a solution containing a microorganism; (ii) acoustically treating the solution with sound waves having a sine waveform at a preselected frequency and at a power intensity of less than 10 W/cm² to produce a minimum 10% increase in microorganism concentration compared to a non-acoustically treated solution for a same treatment time, wherein the solution is acoustically treated in a vessel having a volume of at least 12.5 L; and (iii) optionally isolating the acoustically-treated microorganism.

### FIELD OF INVENTION

The present invention relates to a method and device of improving at least one of the growth and metabolic rate of a microorganism.

The present invention also relates to a method and a device of culturing a microorganism. The present invention relates particularly, although by no means not exclusively, to a method and a device of culturing yeast with an improved growth rate.

The present invention also relates to a method and a device of producing a microorganism-fermented product, such as a microorganism-fermented food product, industrial enzymes, biofuel and manufactured yeast. The present invention relates particularly, although by no means exclusively, to a method and a device of producing beer with reduced fermentation time.

### BACKGROUND OF INVENTION

Fermentation is a key process in the production of many food products such as bread, cheese, beer, wine, spirits, tea, cider and yoghurt. The fermentation process typically involves the use of a microorganism such as yeast, bacteria and fungi to convert carbohydrates in one or more of the feed material into alcohol, carbon dioxide, organic acids and other metabolites to form the food product.

In beer brewing, the fermentation step involves the conversion of utilisable carbohydrates, such as glucose, in wort (typically comprising malted barley, hops and water) by yeast to ethyl alcohol and carbon dioxide. Similarly in biofuel production, yeast, such as Saccharomyces Cerevisiae, is utilised to ferment sugars into bioethanol, and in detergent production, bacteria such as Bacillus subtilis or Bacillus licheniformis is utilised to commercially manufacture enzymes.

Over the years, brewers have sought to increase production output by reducing fermentation time or increasing the fermentation rate which are typically dictated by the concentration and activity of the microorganism involved in the breakdown of the carbohydrates.

Some brewers have attempted to achieve this by controlling the type and amount of nutrients and additives added during fermentation. One disadvantage of this approach is that the additional substances can adversely impact the quality (e.g. flavour profile) of the final food product. Any introduced additives may also pose an allergenic risk to some consumers.

It is therefore desirable to develop a method of improving the reproduction or growth rate of the microorganism before or during the fermentation process. More desirably, the increase in reproduction or growth rate of the microorganism is achieved without adversely impacting the quality of the food product. Even more desirably, the increase in reproduction or growth rate of the microorganism is achieved without altering the recipe of the food product.

It is also desirable to develop a device that is configured to improve the reproduction or growth rate of the microorganism before or during the fermentation process. More desirably, the device can be retrofitted to existing microorganism culturing or fermentation equipment.

### SUMMARY OF INVENTION

The present invention provides a method and an acoustic device of acoustically treating a microorganism to improve its growth rate. Suitably, the present invention may improve yeast propagation, and/or increase the amount of biomass generated by the process.

The method and acoustic device may be used to treat a microorganism prior to the microorganism being used to ferment a food product or may be used to treat a microorganism during an in-situ fermentation process.

In this specification, the term “acoustic device” encompasses any device that includes a speaker and a signal generator. The signal generator delivers a signal to the speaker to generate sound from the speaker.

The signal generator may be configured to deliver signals having different waveforms. Suitably, the signal generator is configured to deliver a signal having a sine waveform.

In this specification, the term “biomass” encompasses any organic material generated during the process including microbes.

The treatment method includes a step of acoustically treating the microorganism with sound waves having a sine waveform at a preselected frequency. Suitably, the sound waves are also delivered at a power intensity of less than 10 W/cm² . It can be appreciated that sound waves having a power intensity greater than 10 W/cm² may be used in some embodiments.

Different frequency ranges affect cell propagation differently. These effects are influenced by a combination of factors, including acoustic pressure. This applies across various frequencies, where both stable and inertial cavitation may occur.

Significantly, the Applicant noted that acoustic treatment of a microorganism with sound waves ranging from 20,000 to 100,000 Hz causes cell lysis. It is believed that this phenomenon is due to inertial cavitation which may be caused by the violent collapse of bubbles. This generates intense shear forces on the microorganism and can lead to the rupture of cell membranes and the formation of reactive species that may result in cellular death.

In one embodiment, the treatment method includes a step of acoustically treating the microorganism with sound waves at a frequency not exceeding 2,000 Hz, suitably the ranging from 60-2,000 Hz.

In another embodiment, the treatment method includes a step of acoustically treating the microorganism with sound waves at a frequency of at least 750,000 Hz, suitably ranging from 750,000-3,000,000 Hz. More suitably, the treatment method includes a step of acoustically treating the microorganism with sound waves at a frequency ranging from 750,000-2,500,000 Hz.

The treatment method may include a step of generating standing waves, suitably surface standing waves, for treating the microorganism. In this specification, surface standing waves are confined to the interface of the air and microorganism-containing medium.

It can be appreciated that the microorganism is typically contained in a suitable vessel.

The method and acoustic device of the present invention are suitably for commercial-scale operations. Typically, the vessel has a volume that ranges from 12.5 L to any commercial-scale volume. Typically, vessels used in commercial scale operations have a volume ranging from about 100 L to about 400,000 L.

The standing waves may be generated by positioning the acoustic device on a microorganism-containing vessel to transmit sound waves that reflect off a surface such as an internal wall of the vessel. Suitably, the acoustic device is positioned as close as possible to the microorganism. This may involve positioning the acoustic device in close proximity to a yeast crop in the vessel.

In one embodiment, the treatment method includes positioning a microorganism-containing vessel under an acoustic device such that the acoustic device transmits sound waves that travel downwardly through a microorganism-containing medium and reflect off the internal base surface of the vessel to generate standing waves in the microorganism-containing medium.

The treatment method may include positioning a microorganism-containing vessel between an opposing pair of acoustic devices which generate sound waves having the same wavelength and frequency that interfere with each other, and generate standing waves in the microorganism-containing medium in the vessel.

The treatment method may include positioning a vessel containing the microorganism above an acoustic device which transmits low frequency vibrations not exceeding 2,000 Hz to the vessel and generates Faraday waves in the microorganism-containing medium. Typically, the low frequency vibrations do not exceed the natural frequency of the microorganism. Beneficially, it was observed that Faraday waves improve mass transfer in the microorganism-containing medium.

The treatment method may include positioning a vessel containing the microorganism above an acoustic device which transmits high frequency vibrations of at least 750,000 Hz to the vessel and generates Faraday waves in the microorganism-containing medium.

It was observed that the acoustic treatment produced a minimum 10% increase in microorganism concentration compared to a non-acoustically treated microorganism-containing medium/solution for the same treatment time.

The present invention also provides a method of culturing a microorganism with enhanced growth rate by acoustically treating the microorganism. Suitably, the microorganism comprise yeast. More suitably, the microorganism is adapted to produce a yeast-fermented product. In one example, the yeast-fermented product is a food product, typically a beverage, more typically beer. Other examples of yeast-fermented products include biofuels, typically bioethanol, and biological enzymes, typically detergent enzymes.

The method according to the invention may include:
(i) providing a solution containing a microorganism;
(ii) acoustically treating the solution with sound waves having a sine waveform at a preselected frequency and at a power intensity of less than 10 W/cm² to produce a minimum 10% increase in microorganism concentration compared to a non-acoustically treated solution for a same treatment time, wherein the solution is acoustically treated in a vessel having a volume of at least 12.5 L; and
(iii) optionally isolating the acoustically-treated microorganism.

The minimum 10% increase in microorganism concentration does not need to be achieved for the entire duration of the acoustic treatment. That is, the change in microorganism concentration may be less than 10% any time before conclusion of the acoustic treatment.

The vessel may have a volume ranging from about 12.5 L to about 400,000 L.

The solution may be subjected to sound waves at a frequency not exceeding 700 Hz, typically not exceeding 300 Hz, typically not exceeding 200 Hz, typically not exceeding 100 Hz, typically not exceeding 80 Hz. In one embodiment, the solution is subjected to sound waves at a frequency ranging from 400-600 Hz. In another embodiment, the solution is subjected to sound waves at a frequency not exceeding 200 Hz.

The solution may be subjected to sound waves at a frequency of at least 750,000 Hz. Suitably, the solution is subjected to sound waves at a frequency not exceeding 3,000,000 Hz. More suitably, the solution is subjected to sound waves at a frequency ranging from 750,000 to 2,500,000 Hz.

The solution may be subjected to sound waves at the resonant frequency of the solution.

Step (ii) may involve acoustically treating the solution with sound waves at a power intensity not exceeding 8 W/cm², typically 6 W/cm², and typically 4 W/cm². In one embodiment, the solution is subjected to sound waves at a power intensity of about 10⁻⁴ W/m² (~80 dB).

Step (ii) may involve acoustically treating the solution with sound waves having a duty cycle of at least 50%, Suitably, step (ii) may involve acoustically treating the solution with sound waves having a duty cycle of at least 80%, more suitably 85%.

Step (ii) may involve acoustically treating the solution with sound waves having a root-mean-square (RMS) power of at least 5 W. Through their experimental trials, it was determined that acoustically treating the solution with sound waves having an RMS power less than 5 W did not enhance microbial growth rate or enhanced microbial growth with less than 10% increase in microorganism concentration.

Step (ii) may involve acoustically treating the solution with sound waves having an RMS power of at least 5 W. Suitably, step (ii) involves acoustically treating the solution with sound waves having an RMS power of at least 5 W, across one or more acoustic devices.

The method may include determining the number of acoustic devices and their RMS power by the volume of the vessel.

Step (ii) may involve acoustically treating the solution with sound waves having an RMS power of either 5 W or 90 W.

Step (ii) may involve a treatment time of up to 10 days, typically up to 7 days, more typically up to 5 days.

Step (ii) may produce a culture having a minimum 20% increase in microorganism concentration, typically a minimum 30% increase in microorganism concentration, more typically a minimum 40% increase in microorganism concentration, even more typically a minimum 50% increase in microorganism concentration.

The isolating step may include subjecting the acoustically-treated microorganism to solid-liquid separation. Typically, the isolating step involves centrifuging the solution to isolate the microorganism.

The method may produce a culture having a minimum increase in microorganism concentration of 10%, typically of 30%, more typically of 50%, even more typically of 60%, after a treatment time of up to 10 hours.

The method may produce a culture having a minimum increase in microorganism concentration of 10%, typically of 30%, more typically of 50%, even more typically of 60%, after a treatment time of up to 20 hours.

The method may produce a culture having a minimum increase in microorganism concentration of 10%, typically of 30%, more typically of 50%, even more typically of 60%, after a treatment time of up to 30 hours.

The sound waves may generate surface standing waves, suitably Faraday waves in the microorganism-containing medium/solution.

The method may include positioning the acoustic device above a vessel containing the solution such that the acoustic device transmits sound waves that travel downwardly through the solution and reflect off the internal base surface of the vessel.

The method may include positioning a pair of acoustic devices on opposing sides of a vessel containing the solution, wherein the pair of acoustic devices generate sound waves with the same wavelength and frequency that interfere with each other, and generate standing waves in the solution.

The method may include positioning an acoustic device under a vessel containing the solution, wherein the acoustic device transmits low frequency vibrations and generates surface standing waves in the form of Faraday waves in the solution. Typically, the low frequency vibrations do not exceed the natural frequency of the solution containing the microorganism.

The sound waves may be transmitted continuously for the duration of the treatment time. Alternatively, the sound waves are pulsed for the duration of the treatment time.

The sound waves may be transmitted at a dB Sound Pressure Level (SPL) not exceeding 150 dB, typically not exceeding 100 dB, more typically not exceeding 80 dB, even more typically about 80 dB.

The method may include maintaining the solution at a temperature not exceeding 50° C., typically not exceeding 40° C., typically not exceeding 30° C., typically not exceeding 20° C.

The microorganism may be a eukaryote. Typically, the microorganism is yeast.

The present invention also provides a method and an acoustic device of producing a yeast-fermented food product with reduced fermentation time by applying the previously described acoustic treatment to one or more microorganism-inoculated precursors of the food product. In one example, the food product is a beverage.

In this specification, the term “precursor” refers to any ingredient or combination of ingredients such as wort or extract used in the making of the food product.

Whilst the present invention is particularly suitable for beer brewing, it is equally applicable to the preparation of other yeast-fermented food products such as wine, cider, kombucha and wash.

The method of producing a yeast-fermented food product according to the invention may comprise:
(i) providing a precursor of the food product;
(ii) inoculating the precursor with a microorganism to form an inoculate;
(iii) acoustically treating the inoculate with sound waves having a sine waveform at a preselected frequency and at a power intensity of less than 10 W/cm² to produce a minimum 10% reduction in fermentation time compared to a non-acoustically-treated inoculate and form a fermentate, wherein the inoculate is acoustically treated in a vessel having a volume of at least 12.5 L; and
(iv) optionally removing the yeast from the fermentate and forming the fermented food product.

The present invention also provides a method of producing a yeast-fermented beverage, the method comprising:
(i) providing a fermentable wort;
(ii) inoculating the fermentable wort with a microorganism to form an inoculate;
(iii) acoustically treating the inoculate with sound waves having a sine waveform at a preselected frequency and at a power intensity of less than 10 W/cm² to produce a minimum 10% reduction in fermentation time compared to a non-acoustically-treated inoculate and form a fermentate, wherein the inoculate is acoustically treated in a vessel having a volume of at least 12.5L; and
(iv) optionally removing the yeast from the fermentate and forming the fermented beverage.

The vessel may have a volume ranging from about 12.5 L to about 400,000 L.

Fermentation time or rate may be determined by monitoring the alcohol content of the fermentate. This may be achieved by measuring any one or more of Degrees Plato (°P), specific gravity, sugar content, yeast cell population and pH of the fermentate.

Step (i) may include subjecting a solution of fermentable wort precursors (e.g. malted barley and hops) to an acoustic treatment prior to step (ii). The Applicant discovered that acoustic treatment of the fermentable wort precursors may enhance enzymatic activity during wort production.

Step (i) may include maintaining a solution of the fermentable wort precursors or fermentable wort at a temperature not exceeding 80° C., typically not exceeding 70° C., typically about 66° C. More suitably, the temperature is maintained for at least 30 minutes, typically for at least one hour.

The selected temperature and holding time may be dictated by the flavour profile of the fermented beverage. For example, it was observed that temperatures ranging from 50-65° C. form carbohydrates which can be utilised (broken down) by the yeast whereas temperatures above 60° C., more specifically ranging from 69° C. to 77° C., typically form longer-chained carbohydrates which are less or not utilisable by the yeast.

Step (i) may include subjecting the fermentable wort to liquid/solid separation to remove trub from the wort. The trub-free fermentable wort may be heated to a temperature of at least 100° C. Suitably, the trub-free fermentable wort is heated for at least 30 minutes, typically for at least 1 hour. The heated fermentable wort may be cooled to a temperature not exceeding 40° C., typically not exceeding 30° C., typically not exceeding 20° C.

Step (ii) may involve inoculating the fermentable wort precursor or wort with a microorganism at a concentration of at least 5 million cells/mL, typically at least 6 million cells/mL, more typically at least 7 million cells/mL, even more typically 7.2 million cells/mL. Suitably, step (ii) involves inoculating the fermentable wort precursor or wort with a microorganism at a concentration of at least 5 million cells/mL, typically at least 6 million cells/mL, more typically at least 7 million cells/mL, even more typically 7.2 million cells/mL to produce ale beer.

Step (ii) may involve inoculating the fermentable wort precursor or wort with a microorganism at a concentration of at least 10 million cells/mL, typically at least 12 million cells/mL, more typically at least 14 million cells/mL, even more typically 14.4 million cells/mL to produce lager beer.

The method may include a step of homogenising the inoculate. Typically, the inoculate is homogenised by in-line dosing.

The fermentable wort may comprise a mixture of malt grist and water at a liquor to grist ratio ranging from 2.5 to 4:1, typically 2.8:1.

Any commercial lager and ale yeast strain capable of fermenting wort may be used with the present invention. Suitable yeast strains include Saccharomyces pastorianus and Saccharomyces cerveisiae. Suitable commercially available yeast product are Bluestone yeast LNDN/WC™, Fermentis US05, Fermentis US04 and Fermentis Saflager W-34/70.

The standing waves may be formed by positioning the inoculate between an opposing pair of acoustic devices, and having the acoustic devices generate sound waves travelling in opposite directions at the same wavelength and frequency, and which interfere with each other. Alternatively, the standing waves may be formed by positioning the inoculate between one acoustic device and a reflective surface, such as a plate.

In another embodiment, the standing waves, typically in the form of Faraday waves, are formed by positioning the inoculate above an acoustic device which transmits low frequency vibrations to the inoculate. Typically, the low frequency vibrations do not exceed the natural frequency of the inoculate.

In yet another embodiment, the standing waves are formed by positioning a vessel containing the inoculate under an acoustic device such that the acoustic device transmits sound waves that travel downwardly through the inoculate and reflect off the internal base surface of the vessel.

Step (iii) may involve maintaining a solution at a temperature ranging from 15-30° C., suitably 17-24° C. This temperature range is particularly suitable for producing ale beer.

Step (iii) may involve maintaining a solution at a temperature ranging from 10-15° C., suitably 8-14° C. This temperature range is particularly suitable for producing lager beer.

Step (iii) may involve a treatment time of up to 10 days, typically up to 7 days, more typically up to 5 days.

Step (iii) may involve a treatment time of up to 80% of the fermentation time, typically up to 60% of the fermentation time, more typically up to 40% of the fermentation time.

The sound waves may be transmitted continuously for the duration of the treatment time. Alternatively, the sound waves are pulsed for the duration of the treatment time.

The inoculate may be subjected to sound waves at a frequency not exceeding 500 Hz, typically not exceeding 300 Hz, typically not exceeding 200 Hz, typically not exceeding 100 Hz, typically of about 80 Hz.

The inoculate may be subjected to sound waves at a frequency of at least 750,000 Hz. Suitably, the inoculate is subjected to sound waves at a frequency not exceeding 3,000,000 Hz. More suitably, the inoculate is subjected to sound waves at a frequency ranging from 750,000 to 2,500,000 Hz.

The inoculate may be subjected to sound waves at the resonant frequency of the inoculate.

The inoculate may be subjected to sound waves at a power intensity not exceeding 8 W/cm², typically not exceeding 6 W/cm², and more typically not exceeding 4 W/cm². In one embodiment, the inoculate is subjected to sound waves at a power intensity of about 10⁻⁴ W/m².

The sound waves may be transmitted at a dB Sound Pressure Level (SPL) not exceeding 150 dB, typically not exceeding 100 dB, more typically about 80 dB.

The inoculate may be subjected to sound waves having a duty cycle of 50%. Suitably, the inoculate is subjected to sound waves having a duty cycle of at least 80%, typically 85%.

The inoculate may be subjected to sound waves having an RMS power of at least 5 W.

The inoculate may be subjected to sound waves having an RMS power of at least 5 W, across one or more acoustic devices producing the sound waves. Suitably, the inoculate is subjected to sound waves having an RMS power of either 5 W or 90 W.

The method may include a step of filtering the fermented beverage.

Step (iii) may produce a minimum 20% increase in fermentation rate, typically a minimum 30% increase in fermentation rate, more typically a minimum of 40% increase in fermentation rate, even more typically a minimum of 50% increase in fermentation rate.

The increase in fermentation rate can be determined by measuring the change in specific gravity or pH of the fermentate.

Step (iii) may produce a minimum 20% reduction in total fermentation time, typically a minimum 30% reduction in total fermentation time, more typically a minimum of 40% reduction in total fermentation time, even more typically a minimum of 50% reduction in total fermentation time.

The reduction in fermentation time can be determined by measuring the time taken to achieve complete fermentation.

In this specification, complete fermentation involves at least fermentation of 60 wt %, typically 60-95 wt %, more typically 70-80 wt % of the inoculate. This may be determined by measuring the Degrees Plato °P of the fermentate. A predetermined green beer target Degrees Plato °P may be set prior to fermentation. Fermentation is considered complete when the target Degrees Plato °P is reached.

Step (iv) may include subjecting the fermentate to solid-liquid separation. Typically, step (iv) involves centrifuging the fermentate to isolate the microorganism.

Step (iv) may include acoustically treating the isolated microorganism to rejuvenate the microorganism.

The method may include a step of filtering the fermented beverage.

The present invention also provides an acoustic device capable of performing the acoustic treatments described previously. Suitably, the acoustic device is retrofittable to existing fermenters or microorganism culturing tanks. More suitably, one or more acoustic devices may be utilised.

The acoustic device according to the present invention may comprise:
a housing having a first end that is configured to receive a sound generator and a second end including a connector that is connectable to a fermenter or microorganism culturing tank having a volume of at least 12.5 L; and
a sound generator located within the housing configured to transmit sound waves having a sine waveform at a predetermined frequency and at a power intensity of less than 10 W/cm² into the fermenter or microbial culturing tank.

The housing may be waterproof. Typically, the housing is corrosion-resistant.

The first end includes a cover to retain the sound generator within the housing.

The connector may comprise a cap that is shaped to connect with an appropriate fitting on a fermenter or microorganism culturing tank. Suitably, the cap comprises a dome-shaped top with a circumferential rim.

The sound generator may be any device capable of producing a sound. Suitable examples of sound generators include portable radios, audio and media players, speakers and transducers. The sound generator may be controllable wirelessly via any suitable means, for example a mobile network, the internet, a satellite network, WI-FI, Ethernet, Bluetooth®, infrared, etc.

The housing may be structured to impart vibrations to the fermenter or microorganism culturing tank. Suitably, the housing is made of a metallic material. More suitably, the metallic material is stainless steel.

The housing may have a frustoconical body. The body may comprise a rotund central portion that decreases in diameter towards the first and second ends. Through their research, the Applicant discovered that the frustoconical shape of the housing body facilitates transmission of the sound waves.

The first end may have an opening to receive the sound generator.

The opening at the first end may have a diameter that is larger than the diameter of the connector.

The housing may include an attachment means that allow the acoustic device to be secured to an inner surface of a fermentation tank. Alternatively, the acoustic device may float within the fermentation tank. In a further alternative embodiment, the acoustic device is embedded within the tank wall. For example, the tank may have a multi-layered wall structure with the acoustic device positioned between the outermost and innermost layers.

The sound generator may also be fixed to an external wall of the fermentation tank.

### BRIEF DESCRIPTION OF THE DRAWINGS

FIG. 1 is a graph illustrating the variation in the cell concentration of yeast in a yeast-inoculated malt extract acoustically treated with sound at 80 Hz, 80 dB, SPL, 20 μPa according to one form of the invention compared to a silent control.

FIG. 2 is a graph illustrating the variation in the cell concentration of yeast in a yeast-inoculated malt extract acoustically treated with sound at 100 Hz, 80 dB, SPL, 20 μPa according to one form of the invention compared to a silent control.

FIG. 3 is a graph illustrating the variation in °P of an inoculate acoustically treated with instrumental playlist broad band music ranging from 80-700 Hz, 80 dB, SPL, 20 μPa according to one form of the invention compared to a silent control

FIG. 4 is a graph illustrating the variation in °P of an inoculate acoustically treated with single tone sound at 80 Hz, 80 dB, SPL, 20 μPa compared to a silent control.

FIG. 5 is a graph illustrating the variation in °P of an inoculate acoustically treated with single tone sound at 500 Hz, 80 dB, SPL, 20 μPa compared to a silent control.

FIG. 6 is a graph illustration the variation in °P of an inoculate acoustically treated with single tone sound at 80 Hz, 80 dB, SPL, 20 μPa having a triangle waveform, compared to a silent control.

FIG. 7 is a graph illustration the variation in °P of an inoculate acoustically treated with single tone sound at 80 Hz, 80 dB, SPL, 20 μPa having a square waveform, compared to a silent control.

FIG. 8 is a graph illustration the variation in °P of an inoculate acoustically treated with single tone sound at 80 Hz, 80 dB, SPL, 20 μPa having a sawtooth waveform, compared to a silent control.

FIG. 9 is a graph illustrating the variation in °P of an inoculate acoustically treated with single tone sound at 80 Hz, 80 dB having a sine waveform at a fermentation temperature of 12.5° C. to 13.5° C., compared to a silent control.

FIG. 10 is a graph illustrating the variation in °P of an inoculate acoustically treated with single tone sound at 80 Hz, 80 dB having a sine waveform at a duty cycle of 80%, at a fermentation temperature of 18° C. to 22° C., compared to a silent control.

FIG. 11 is a graph illustrating the variation in °P of an inoculate acoustically treated with single tone sound at 80 Hz having a sine waveform, compared to a silent control.

FIG. 12 is a graph illustrating the variation in °P of an inoculate acoustically treated with single tone sound at 80 Hz having a sine waveform at a fermentation temperature of 21° C. compared to a silent control.

FIG. 13 is a graph illustrating the effect of acoustic treatment on pH of the process solution.

FIG. 14 is a graph illustrating the effect of acoustic treatment on yeast cell count.

FIG. 15 depicts a perspective view of an acoustic device according to one form of the invention.

### DETAILED DESCRIPTION

In the following detailed description, reference is made to accompanying figures which form a part of the detailed description. It will be readily understood that the aspects of the present disclosure, as generally described herein and illustrated in the figures may be arranged, substituted, combined, separated and designed in a wide variety of different configurations, all of which are contemplated in this disclosure.

Whilst the examples provided are directed towards improving the growth rate of yeast with respect to the brewing of beer, the present invention is equally applicable to the preparation of other yeast-fermented products such as yeast-fermented food products (e.g., wine and cider), industrial enzymes, biofuel and manufactured yeast. The present invention is also applicable to other microorganisms such as bacteria and fungi.

Through research and experimentation, the Applicant has identified parameters of the acoustic treatment including frequency range and intensity of the sound waves that can increase biochemical reactions in the yeast while minimising cell lysis.

Cell lysis is dependent on various factors such as frequency, power intensity and acoustic cavitation. For example, acoustic cavitation leads to formation of microbubbles, when upon collapse, generate intense localised shear forces capable of disrupting cell membranes, leading to cell lysis.

Without being bound by theory, it is believed that the identified acoustic parameters increase permeability of the cell membrane by imparting controlled mechanical, particularly shear forces to stimulate metabolism and growth of the yeast cells. This allows for efficient uptake of nutrients and waste elimination. It is also believed that the identified frequency ranges can induce resonant vibrations and trigger biochemical reactions. This was discovered to lead to an increase in the growth rate of the yeast and reduction in fermentation time.

In this respect, microbial growth typically contains four stages: lag phase, exponential phase, stationary phase and death phase. The lag phase is an adaptation period of slow growth where the microorganisms adjust to their environment.

The exponential phase is a period of rapid growth when the microorganisms have generated sufficient enzymes and accumulated sufficient nutrients. The stationary phase is a period of plateauing microbial population where there are insufficient nutrients to sustain microbial growth. The death phase is a period of declining microbial population where one or more nutrients is exhausted or there is accumulation of inhibitory products. The enhanced uptake of nutrients and waste elimination provided by the acoustic treatment of the present invention may reduce the lag phase and allow the microorganisms to enter the exponential phase more quickly.

The present invention provides a method of acoustically treating a microorganism, particularly but not exclusively limited to yeast, to increase the growth rate of the microorganism by at least 10% compared to a non-acoustically treated solution for a same treatment time.

The increase in growth rate also results in an increase in biomass production which is due at least in part to the increased microbial population.

Enhancing biomass production has a number of benefits. For example, yeast biomass is a significant by-product in the beverage industry as it is attributed to rich nutritional profile typically comprising high protein and vitamin content.

The enhanced biomass production can be determined by measuring various parameters such as microbial cell count, microbial activity or change in microbial mass.

The Applicant observed in some of their trials that acoustically treating the microorganism with sound waves at a frequency exceeding 2,000 Hz would adversely impact cell activity and often leads to cell lysis usually at a frequency above 15 kHz. It was also observed in some instances that power intensity influenced cell lysis.

In contrast, the Applicant demonstrated that at a frequency below 2,000 Hz, suitably at a frequency ranging from 60 to 2,000 Hz, the sound waves promote stable cavitation, where bubbles oscillate without collapsing violently. This can enhance mixing and potentially stimulate yeast cells to enhance permeability.

Surprisingly, it was also observed that increasing the frequency further, particularly to a range from 750,000 to 2,500,000 Hz, enabled the method to revert to a non-destructive mode and promote cell proliferation. Without being bound by theory, it is believed that this phenomenon is the result of a reduction in the size of the bubbles at this high frequency range. At this frequency range, bubbles possessing stable oscillations and predominantly stable cavitation are formed which reduces shear forces on the cells.

Sound waves having different waveforms including triangle, square and sawtooth were tested.

A sine waveform consists of the fundamental frequency, without any harmonics. The spectrum of a sine waveform contains a single peak at the fundamental frequency and importantly, there are no additional harmonics present. A square waveform consists of a fundamental frequency and odd-numbered harmonics (e.g., 3^{rd}, 5^{th} and 7^{th}) that decrease in amplitude with increasing frequency. The amplitudes of the harmonics decrease with a rate of 1/n, where n is the harmonic number. A sawtooth waveform contains all even and odd harmonics. The amplitudes of the harmonics for a sawtooth waveform decreases inversely, with a rate of 1/n where n is the harmonic number. A triangle waveform contains only odd-numbered harmonics. In a triangle waveform, the amplitudes of the harmonics decrease with a rate of 1/n², where n is the harmonic number.

Experimentation by the Applicant identified sound waves having sine waveform as providing optimal increases in microorganism concentration.

A skilled person would appreciate that typical acoustic devices such as sound speakers produce a variety of complex waveforms comprising a mixture of different frequencies and harmonics. As a result, they may inherently produce some level of waveform distortion, resulting in a complex signal output. In contrast, the present invention utilises a signal generator that enables the precise generation of pure sine waves.

In some experiments, the Applicant also observed that sound waves having a duty cycle of at least 80% and RMS of at least 5 W further enhanced fermentation rates.

Experimentation by the Applicant identified two low frequency ranges as providing optimal increases in microorganism concentration. The first frequency range is less than 200 Hz and the second frequency range is from 400-600 Hz.

At a frequency of about 500 Hz, it was also observed that the time taken to complete fermentation of the acoustically treated sample reduced by at least 40%. In one example, the acoustically treated sample achieved completed fermentation in 4 days whereas the control failed to achieve complete fermentation even after 6 days.

The Applicant has developed a number of ways to generate sound waves, particularly standing waves, for the treatment process.

In a first example, a vessel containing the yeast is located between a pair of acoustic devices mounted onto an external surface of the vessel in a diametrically opposed arrangement. The acoustic devices are used to generate sound waves travelling in opposite directions at the same wavelength and frequency which interfere with each other to form standing waves in the yeast-containing liquid in the vessel.

In a second example, one acoustic device, in the form of a vibrating mechanical oscillator such as a sound speaker, is mounted to an underside of a vessel containing the yeast. The device delivers low frequency vibrations, which do not exceed the natural frequency of the yeast-containing solution, upwardly through the vessel and form Faraday waves in the yeast-containing liquid in the vessel.

In these examples, the generated sound waves travel directly through the liquid contained in the vessel.

In a third example, one acoustic device is positioned above a yeast-containing vessel and delivers sound waves downwardly through the vessel. In this example, the sound waves travel through air in the headspace of the vessel and into the liquid contained in the vessel to form standing waves in the yeast-containing liquid in the vessel.

The Applicant has utilised vessels that have a volume of at least 12.5 L in these examples to demonstrate the scalability of the present invention. It is envisaged that the invention is equally deployable to any commercial-scale vessels having a volume of up to 400,000 L.

The acoustic device used in these examples comprises a portable audio player contained inside a waterproof stainless-steel housing. These devices are equally applicable for generating sound waves of different characteristics by changing one or more of their location on the vessel, wavelength and frequency of the sound.

After the acoustic treatment, the treated yeast is isolated by filtration.

The invention also provides a method of producing a yeast-fermented food product in the form of beer. The method includes inoculating wort with yeast typically in a fermentation vessel to form an inoculate.

The inoculate is then treated with sound waves to reduce fermentation time or increase fermentation rate.

The sound waves were transmitted using an acoustic device in the form of an audio speaker mounted under the fermentation tank, at a frequency not exceeding 2,000 Hz sound at a power intensity of less than 10 W/cm² and at an SPL not exceeding 80 db. The audio speaker transmitted low frequency vibrations that do not exceed the natural frequency of the inoculate in the vessel to generate Faraday waves.

Subjecting the inoculate to the acoustic treatment increases the yeast concentration and fermentation rate during the fermentation process. Apart from increased production output, increasing fermentation rate or reduced fermentation time also provides a number of other benefits including improved beer quality (e.g. sensory attributes such as smell and taste). This is because the fermentation process terminates earlier due to greater cellular density, and reduces the amount of undesirable by-products generated by the yeast. It was also discovered that acoustic treatment of the yeast during the fermentation process may increase flocculation. This may enable the spent yeast to be more easily isolated from the fermentate.

The invention also provides an acoustic device for performing the acoustic treatment. The acoustic device may be configured for use with existing microorganism culturing or fermentation equipment.

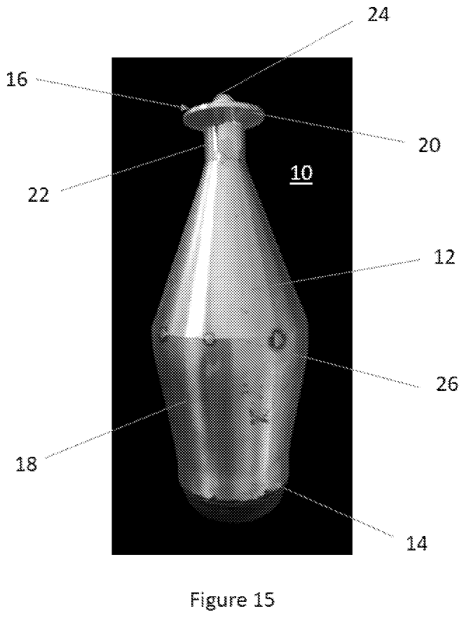

The acoustic device labelled as 10 in FIG. 15 comprises a housing 12 made of a metallic material, typically stainless steel. The housing has a first end 14 and a second end 16 Separated by a body portion 18.

The first end 14 has a circular opening to receive a sound generator (not shown) which is typically a portable audio player that is controllable wirelessly via a mobile network or WIFI.

The sound generator is configured to transmit sound waves at a frequency not exceeding 2,000 Hz at a power intensity of less than 10 W/cm² into the fermenter or microbial culturing tank.

The first end further includes a cover that is positionable to form a water-resistant seal to encase the sound generator in the housing 12.

The second end comprises a cylindrical portion 22 extending from the body 18 and ending in a connector in the form of cap 24. The cap 24 is hat-shaped and comprises a domed head with a circumferential rim 20. The cap 24 is connectable to a fermenter or microorganism culturing tank. In one example, a gasket and ring clamp can be used to engage the rim 20 with an appropriate fitting on a fermentation tank.

The body portion 18 has a frustoconical shape with a rotund central portion 26 that decreases in diameter towards the first and second ends.

The cylindrical portion 22 has a smaller diameter than the first end 14, typically about three times smaller.

Whilst the embodiment shown in FIG. 15 depicts an acoustic device with the above described features, it is envisaged that acoustic devices of other shapes and forms may be used without departing from the broad scope of the present invention.

The sound generator (not shown) generates sound waves having a RMS (Root-Mean-Square) power of at least 5 W.

### RESULTS

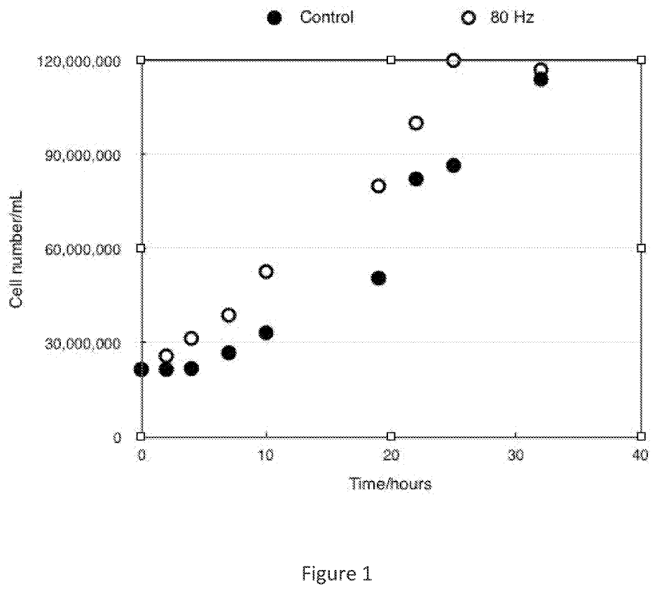

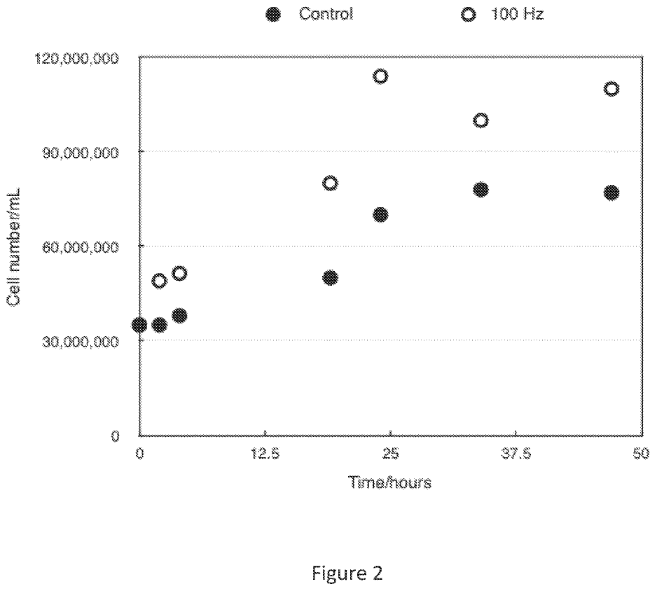

FIGS. 1 and 2 measure the change in yeast concentration in inoculated malt extract with time.

In FIG. 1, yeast (Fermentis us05)-containing malt extract was treated with 80 Hz sound waves for a period of about 30 hours.

The yeast concentration of the acoustically treated sample was observed to increase above the control at around the 3 hour mark with the acoustically treated yeast-containing extract having a concentration of around 2.5×10⁷ cells/mL compared to a concentration of 2.4×10⁷ cells/mL for the control.

This difference was observed to continuously increase with treatment time up to about 20 hours in which the acoustically treated yeast-inoculated extract was observed to have a yeast concentration of 7.5×10⁷ cells/mL compared to a concentration of 5.0×10⁷ cells/mL for the control. This is a 50% increase in yeast concentration.

The yeast concentration of the acoustically treated extract reached its maximum at a concentration of 12×10⁷ cells/mL after around 25 hours. This is compared to a yeast concentration of 8.5×10⁷ cells/mL for the untreated malt extract sample. This is a 42% increase in yeast concentration.

In FIG. 2, yeast (Fermentis us05)-containing malt extract was treated with 100 Hz sound waves for a period of about 50 hours.

The yeast concentration of the acoustically treated sample started to deviate from the control at around the 2 hour mark with the acoustically treated yeast-containing extract having a concentration of around 5.0×10⁷ cells/mL compared to a concentration of 3.2×10⁷ cells/mL for the control. This is a 56% increase in yeast concentration.

The acoustically treated extract continued to maintain an increased yeast concentration over the control with a concentration of 8.0×10⁷ cells/mL at 19 hours compared to 5.0×10⁷ cells/mL for the control, and a 11.2×10⁷ cells/mL concentration at around 25 hours compared to 7.4×10⁷ cells/mL for the control.

At around the 50 hour mark, the acoustically treated sample was observed to have a yeast concentration of 11.0×10⁷ cells/mL compared to 7.5×10⁷ cells/mL for the control. This is a 46% increase in yeast concentration.

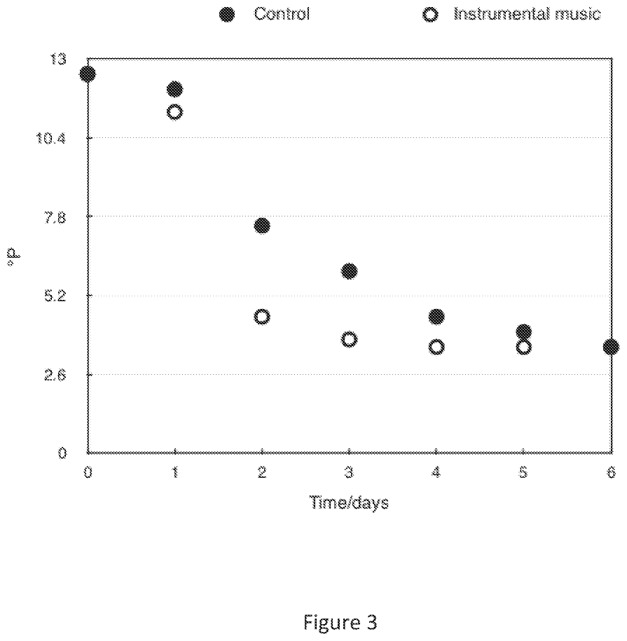

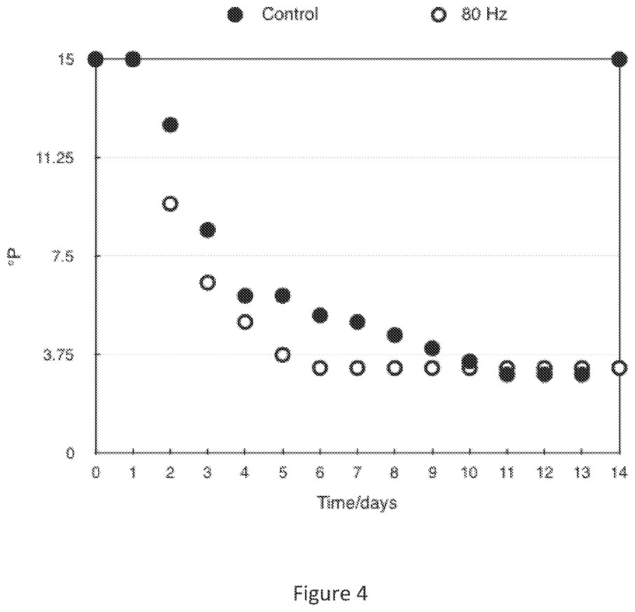

FIGS. 3 and 4 measure the change in Degrees Plato (°P) of the inoculate during fermentation with time. This change corresponds to a change in specific gravity of the inoculate. The change is a measure of the reduction in fermentable carbohydrates, in which 51% is alcohol and 49% is carbon dioxide and can be used to assess the fermentation rate. For these experiments, a predetermined green beer °P of 3.5 was selected to indicate complete fermentation.

In FIG. 3, the inoculate, in the form of yeast-containing wort, was treated with instrumental music playlist broad band music transmitted at 80 dB, SPL, 20 μPa.

At the start of the fermentation process, a minimal difference was observed between the acoustically treated inoculate and the control with the °P reducing from 12.5 to 11.25.

The difference in fermentation rates was maximum at Day 2, where the °P for the acoustically treated inoculate fell to 4.5 compared to a °P of 7.5 for the control.

The °P acoustically treated inoculate subsequently reduced to 3.75 on Day 3 and achieved the target °P value of 3.5 on Day 4. In contrast, the °P of the control reduced to 4.5 on Day 4 and reached the target °P value of 3.5 on Day 6.

In this experiment, fermentation was completed using the acoustically treated inoculate two days (40%) faster than the control.

In FIG. 4, the wort of the present invention was treated with sound delivered at 80 Hz, 80 dB, SPL, 20 μPa.

A change in °P was first detected on Day 2, with a reduction from 15 to 9.5 for the acoustically treated inoculate and a reduction from 15 to 12.5 for the silent control.

The °P of the acoustically treated inoculate reduced to 6.5 on Day 3 and achieved a °P of 3.3 (below the target value of 3.5) on Day 6. In contrast, the °P of the control reduced to 8.5 on Day 3 and only achieved the target value of 3.5 on Day 10.

In this experiment, fermentation time with the acoustically treated inoculate was 50% faster than the control.

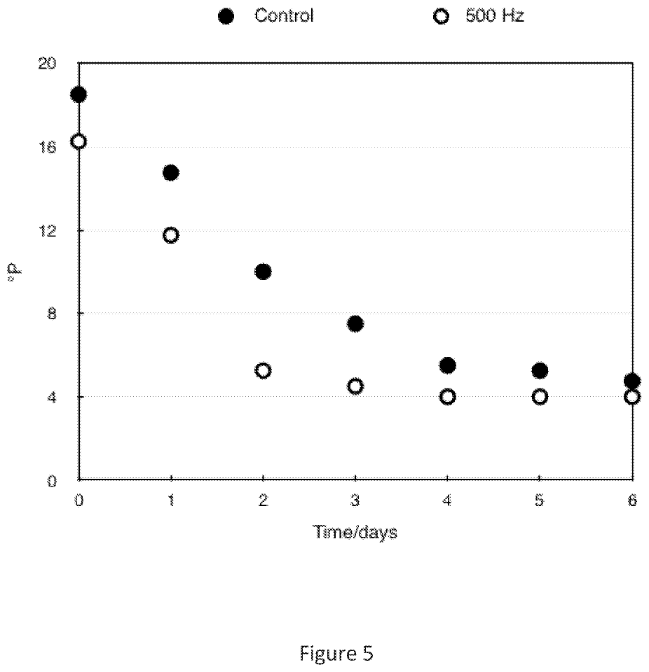

FIG. 5 shows the change in °P of the inoculate during fermentation of ale with time. Complete fermentation was achieved at a green beer °P of 4.

The inoculate was treated with sound delivered at 80 Hz, 80 dB, SPL, 20 μPa.

At the 2 day mark, the acoustically treated inoculate reduced to a °P of about 5 while the control reached a °P of 10.

The target green beer °P of 4 was achieved by the acoustically treated inoculate at the 4 day mark. At the same time, the control achieved a °P just under 6. The control failed to reach the target °P after 6 days. This represents an at least 50% reduction in fermentation time for the acoustically treated inoculate.

The Applicant subsequently performed further experiments to determine the effect of waveform on fermentation rates.

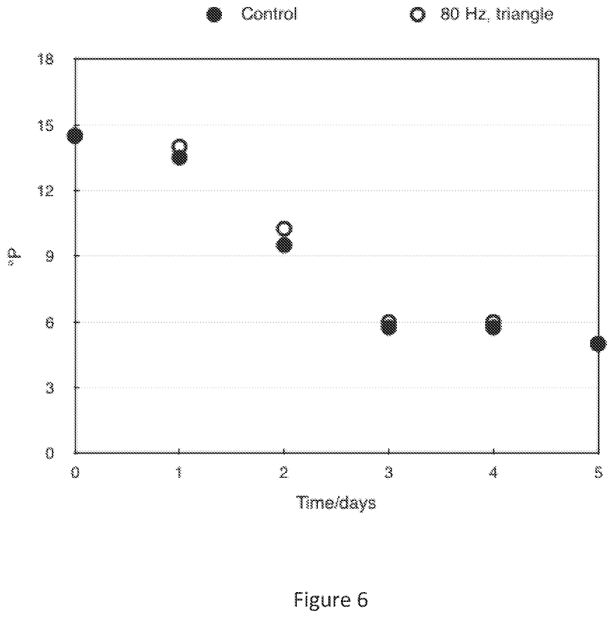

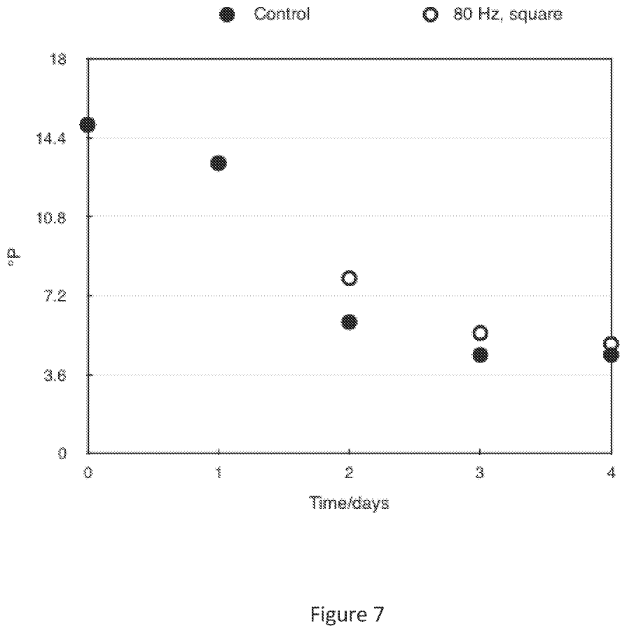

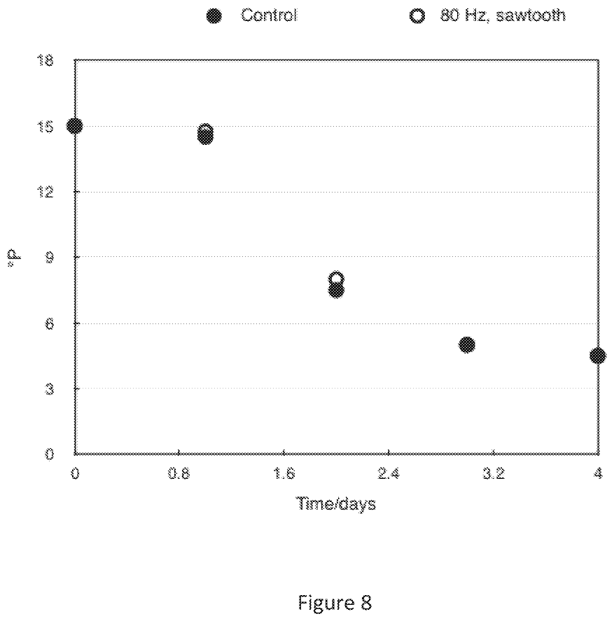

FIGS. 6 to 8 show the change in °P of the inoculate during fermentation of ale with time using sound waves having triangle, square and sawtooth waveforms, respectively, at 80Hz, 80 dB, SPL, 20 μPa. It was observed that the acoustically treated inoculate did not outperform the control in these experiments.

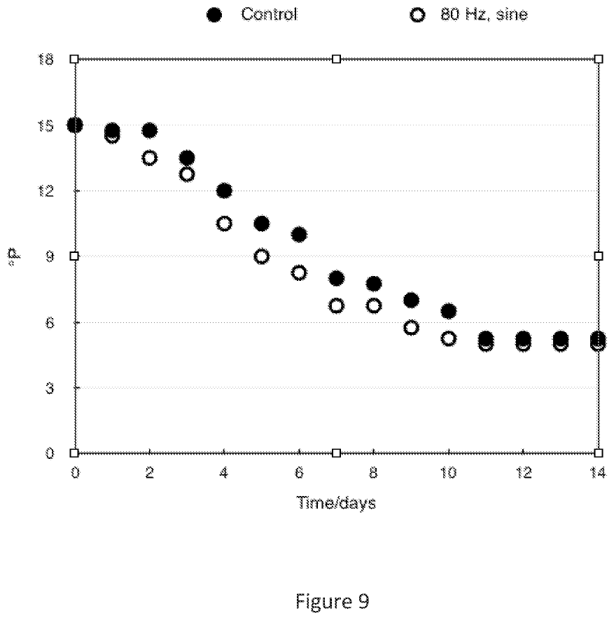

A different outcome was observed when the inoculate was treated with sound waves having a sine waveform. The results are illustrated in FIG. 9.

In FIG. 9, the inoculate is acoustically treated with sound waves having a sine waveform at a fermentation temperature starting at 10.2° C., gradually increasing to 15.8° C. On Day 3, the °P for the acoustically treated inoculate reduced from 15 to 13.5 while a reduction from 15 to 14.8 was only observed for the silent control. Both the acoustically treated inoculate and the silent control reached a °P of 5 on Day 11. From Day 2, the acoustically treated inoculate consistently provided a higher fermentation rate than the control, with a maximum 17% increase in fermentation rate observed on Day 6.

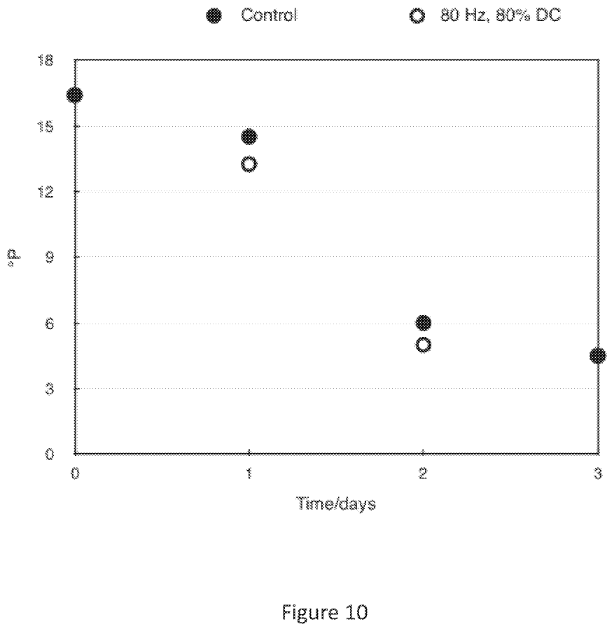

Duty cycle was another parameter that was observed to influence fermentation rate. This parameter was investigated in FIG. 10 which shows the change in °P of the inoculate during fermentation of ale with inoculate acoustically treated with sound waves having a sine waveform and a duty cycle of 80%, at a fermentation temperature at 18° C. to 20° C. In this experiment, the Applicant has found that duty cycle of 80% or more provides an enhanced fermentation rate over the control over 3 days with a minimum 16% increase in fermentation rate during the process.

The target °P of 4.2 was achieved by the acoustically treated inoculate in 2 days while the control achieved the target °P in 3 days. This represents a 33% reduction in fermentation time for the acoustically treated inoculate.

Additionally, the Applicant performed large scale trials to determine whether the enhanced fermentation rate is replicate-able in larger vessels. This would be more representative of commercial operations. In this respect, the experiments relating to FIGS. 6-8 were performed in 12.5 L vessels and the experiments relating to FIGS. 9-10 were performed in 15 L vessels.

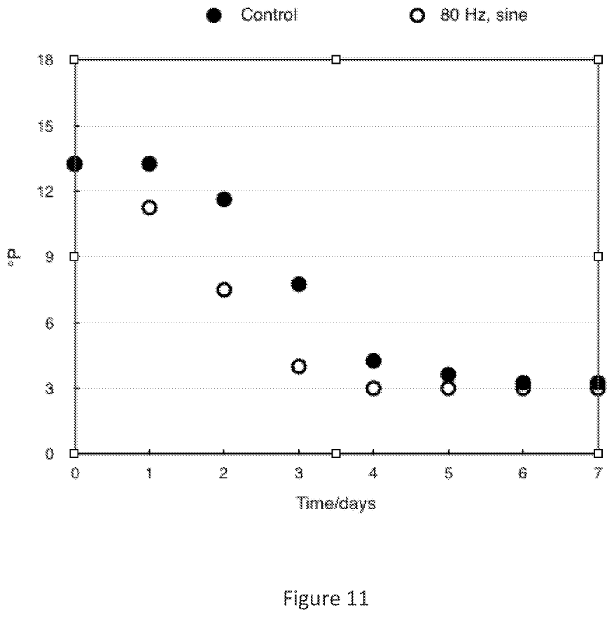

To further investigate the acoustic treatment in scaled up operations, additional experiments were performed on 50 L and 1,000L vessels. The results of these experiments are illustrated in FIGS. 11-14. In these Figures, the inoculate was treated with sound waves having a sine waveform at 80 Hz at a fermentation temperature of 21° C.

FIG. 11 shows the change in °P of the inoculate during fermentation of ale with time. From Day 1, the acoustically treated inoculate was observed to produce a lower °P value compared to the control with a maximum increase in fermentation rate of 40% occurring on Day 2.

The target °P of 3 was achieved by the acoustically treated inoculate at the 4 day mark. The control failed to reach the target °P reaching a lowest °P of 3.25 for the duration of the trial.

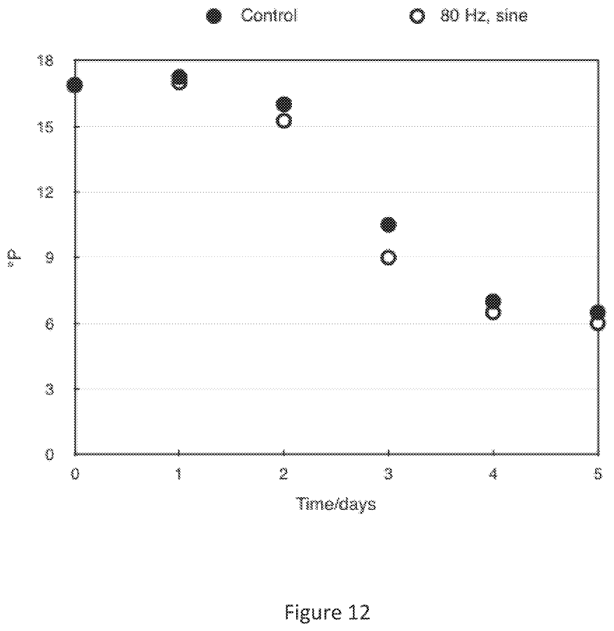

FIG. 12 shows the change in °P of the inoculate during fermentation of ale with time. From Day 1, the acoustically treated inoculate was observed to produce a lower °P value compared to the control with a maximum increase in fermentation rate of 14% occurring on Day 3.

The target °P of 6 was achieved by the acoustically treated inoculate at the 5 day mark. The control failed to reach the target °P within the same time.

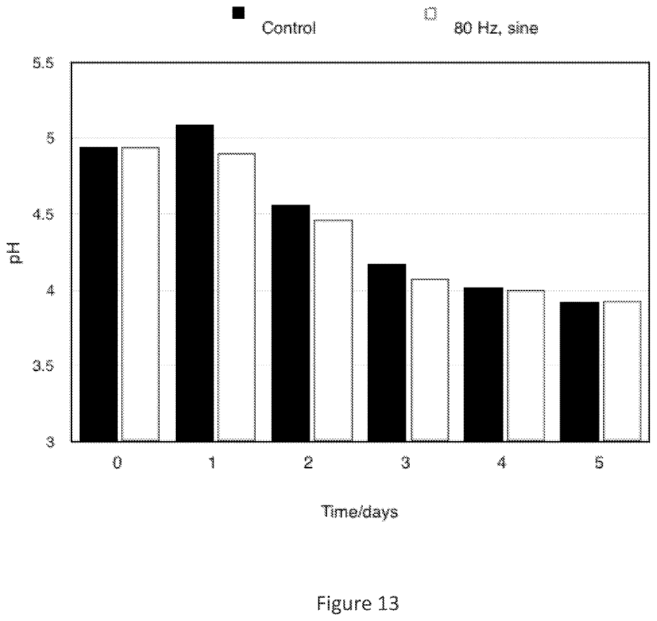

FIG. 13 shows the change in pH of the inoculate during fermentation of ale with time. A skilled person would understand that pH monitoring is a significant factor during fermentation as it enables a better understanding of the yeast activity and the health of the fermentation process. As shown in FIG. 13, the pH of the wort is lower on all days in the acoustically treated vessel compared to the control. This indicates that there is more active fermentation in the acoustically treated vessel as the yeast produces more organic acids.

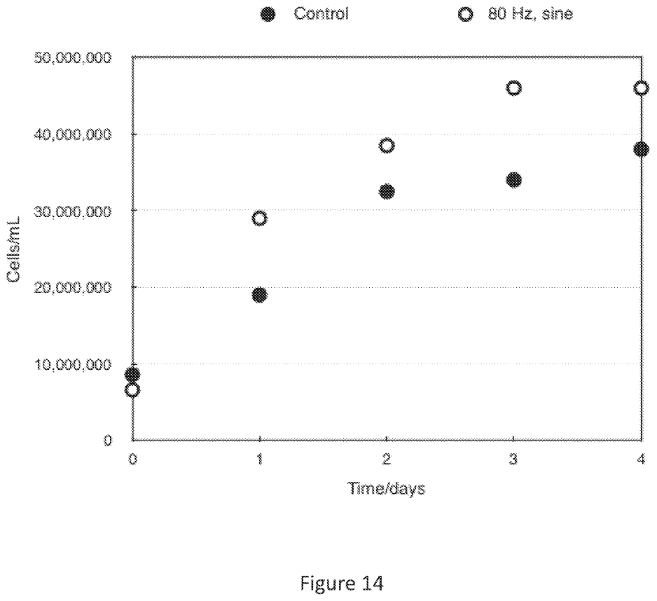

FIG. 14 shows the change in yeast cell count during fermentation of ale with time.

In this experiment, the number of yeast cells in the fermentation vessel treated with sound was initially less than the number of yeast cells in the control vessel. Despite starting with a smaller number of yeast cells, the Applicant observed that the vessel treated with sound generated more yeast cells at a faster rate compared to the control vessel.

Yeast cell concentration of the acoustically treated inoculate increased from 6.6 million cells/mL to 29 million cells/mL on Day 1, 38.5 million cells/mL on Day 2 and 46 million cells/mL on Day 3. The increase of the number of yeast cells generated inside the acoustically treated vessel is significantly higher than the control. A minimum increase in yeast cell number of 18% was observed on Day 2 and a maximum increase in yeast cell number of 35% was observed in Day 3.

As observed in FIG. 14, the increase in the number of yeast cells in the acoustically-treated fermentation vessel indicates that there is high yeast propagation and thus, an increase in biomass, compared to the control. This demonstrates that the present invention is suitable for applications requiring cell proliferation such as enzyme and yeast manufacturing.

The Applicant also discovered that no vicinal diketones (VDKs) were detected in the beer subject to sound treatment during fermentation. Meanwhile, it was found that VDKs were present in the control, indicating that beer subject to sound treatment during fermentation is able to be chilled immediately, whereas the control requires additional time at an elevated temperature to eliminate VDKs.

### Experimental

### 1 Materials

The materials used during the yeast propagation experiments of FIGS. 1 and 2 are set out below.
Brewing yeast: Fermentis us05
Malt extract

The materials and equipment used during the fermentation experiments of FIGS. 3 to 5 are set out below.
50 L tower type pilot brewhouse (wort production)
2×19 L Corni kegs with airlock (fermentation)
Brewing yeast: Bluestone yeast LNDN/WC
Wort (mixture of malted barley, hops, and water)
Acoustic device
Hydrometer for measuring original gravity
Thermometer
pH meter
70 % Ethanol (for sanitising)

The materials and equipment used during the fermentation experiments of FIGS. 6 to 8 are set out below.
12.5 L vessels (for fermentation)
Brewing yeast: Fermentis us05
Acoustic device

The materials and equipment used during the fermentation experiment of FIG. 9 are set out below.
15 L vessels (for fermentation)
Brewing yeast: Fermentis Saflager W-34/70
Acoustic device

The materials and equipment used during the fermentation experiment of FIG. 10 are set out below.
15 L vessels (for fermentation)
Brewing yeast: Fermentis us05
Acoustic device

The materials and equipment used during the fermentation experiment of FIG. 11 are set out below.
50 L vessels (for fermentation)
Brewing yeast: Fermentis us05
Acoustic device

The materials and equipment used during the fermentation experiments of FIGS. 12 to 14 are set out below.
1,000 L vessels (for fermentation)
Brewing yeast: Fermentis us05
Acoustic device
Thermometer
pH meter

### 2 Methods

The methodology for propagating yeast involved acoustically treating the yeast-inoculated malt extract using sound waves transmitted with the following parameters.
80 Hz, 80 dB, SPL, 20 μPa
100 Hz, 80 dB, SPL, 20 μPa

The acoustic device used for the FIGS. 1-11 trials is configured to deliver sound waves having an RMS power of 5 W.

The acoustic device used for the FIGS. 12-14 trials is configured to deliver sound waves having an RMS power of 90 W.

The target wort requirements for FIGS. 3 to 5 are as follows:
Volume: 50 L beer
Wort °Plato: 12
Green beer °Plato: 3.5
Bitterness units: 25
Malt Grist composition: 50% pale, 30% vienna, 10% Abbey, 9% cara munich type 2 and 1% roast barley.
Liquor to grist ratio: 2.8:1.

The target °P requirement for FIGS. 6 to 8 is 4.2.

The target °P requirement for FIG. 9 is 5.

The target °P requirement for FIG. 10 is 4.2.

The target °P requirement for FIG. 11 is 3.

The target °P requirement for FIG. 12 is 6.

The target pH requirement for FIG. 13 is ±4.

The methodology for producing the target beer is described below.

### Preparation of Wort

Wort was prepared by mixing the malt grist with water in a mash vessel at the specified ratio. The mixture was maintained at 66° C. for one hour, followed by liquid/solid separation and boiling for one hour. After this, the wort was cooled to 17.3° C. in a single vessel (Set temperature: 20° C.).

### Inoculation of Yeast

Bluestone yeast LNDN/WC™ yeast was inoculated into the wort at 7.2 million cells/mL. The wort/yeast mixture was recirculated to homogenise the contents and two aliquots of 15 L each were transferred into Corni kegs.

### Fermentation

The Corni keg was placed next to an acoustic device in a fridge operating at 20° C. Sound frequency of 80 Hz, 200 Hz, broadband instrumental music at 80 dB SPL, 20 μPa and silent as control) was investigated. The instrumental music was selected from a Trentmoeller playlist.

The acoustic treatment was maintained for the entire fermentation period. The control Corni keg was placed in another fridge at 20° C. in a separate room with minimal acoustic interference.

### Monitoring

Temperature was monitored regularly with a thermostat and maintained at 20° C. as per standard fermentation husbandry, specific gravity and pH were monitored to indicate the rate/progression of fermentation.

A 100 mL aliquot of wort was taken out per day and gravity of the wort was measured using a sterilised hydrometer to determine the alcohol content. pH of the wort was also measured daily.

It is to be understood that, if any prior art publication is referred to herein, such reference does not constitute an admission that the publication forms a part of the common general knowledge in the art, in Australia or any other country.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A method of culturing a microorganism, the method including:
(i) providing a solution containing a microorganism;
(ii) acoustically treating the solution with sound waves having a sine waveform at a preselected frequency and at a power intensity of less than 10 W/cm² to produce a minimum 10% increase in microorganism concentration compared to a non-acoustically treated solution for a same treatment time, wherein the solution is acoustically treated in a vessel having a volume of at least 12.5 L; and
(iii) optionally isolating the acoustically-treated microorganism.

2. The method according to claim 1, wherein step (ii) includes subjecting the solution to sound waves at a frequency not exceeding 2,000 Hz or at least 750,000 Hz.

3. The method according to claim 1, including subjecting the solution to sound waves at a resonant frequency of the solution.

4. The method according to claim 1, wherein step (ii) includes a treatment time of up to 10 days.

5. The method according to claim 1, wherein the method includes maintaining the solution at a temperature not exceeding 50° C.

6. The method according to claim 1, wherein step (iii) includes subjecting the acoustically-treated microorganism to solid-liquid separation.

7. The method according to claim 1 including transmitting sound waves at a dB Sound Pressure Level (SPL) not exceeding 150 dB.

8. The method according to claim 1, including transmitting sound waves having a duty cycle of at least 50%.

9. The method according to claim 1, including transmitting sound waves having an RMS power of at least 5 W.

10. A method of producing a yeast-fermented food product according to the invention may comprise:
(i) providing a precursor of the food product;
(ii) inoculating the precursor with a microorganism to form an inoculate;
(iii) acoustically treating the inoculate with sound waves having a sine waveform at a preselected frequency and at a power intensity of less than 10 W/cm² to produce a minimum 10% reduction in fermentation time compared to a non-acoustically-treated inoculate and form a fermentate, wherein the inoculate is acoustically treated in a vessel having a volume of at least 12.5 L; and
(iv) optionally removing the yeast from the fermentate and forming the fermented food product.

11. The method according to claim 10, wherein step (iii) includes subjecting the solution to sound waves at a frequency not exceeding 2,000 Hz or at least 750,000 Hz.

12. The method according to claim 10 wherein step (i) includes maintaining a solution of the precursor at a temperature not exceeding 80° C. for at least 30 minutes.

13. The method according to claim 10, wherein step (iii) includes a treatment time of up to 10 days.

14. The method according to claim 10, including subjecting the inoculate to sound waves at a resonant frequency of the inoculate.

15. The method according to claim 10, wherein step (iv) includes subjecting the fermentate to solid-liquid separation to isolate the microorganism.

16. The method according to claim 10, including transmitting sound waves at a dB Sound Pressure Level (SPL) not exceeding 150 dB.

17. The method according to claim 10, including transmitting sound waves having a duty cycle of at least 50%.

18. The method according to claim 10, including transmitting sound waves having an RMS (power of at least 5 W.

19. An acoustic device comprising:
a housing having a first end that is configured to receive a sound generator and a second end including a connector that is connectable to a fermenter or microorganism culturing tank having a volume of at least 12.5 L; and
a sound generator located within the housing configured to transmit sound waves having a sine waveform at a preselected frequency and at a power intensity of less than 10 W/cm² into the fermenter or microbial culturing tank.

20. The acoustic device according to claim 19, wherein the acoustic device is configured to transmit sound waves at a frequency not exceeding 2,000 Hz or at least 750,000 Hz.

21. The acoustic device according to claim 19, wherein the connector comprises a cap comprising a dome-shaped top with a circumferential rim.

22. The acoustic device according to claim 19, wherein the housing has a frustoconical body.
